(19) United States
(12) Patent Application Publication
(10) Pub. No.: US 2010/0266567 A1
(43) Pub. Date: Oct. 21, 2010

(54) USE OF URATE OXIDASE FOR THE TREATMENT OR PROPHYLAXIS OF DISORDERS OR INDIRECT SEQUELAE OF THE HEART CAUSED BY ISCHEMIC OR REPERFUSION EVENTS

(75) Inventors: Wolfgang LINZ, Mainz (DE); Matthias SCHAEFER, Ehringshausen (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 12/715,061

(22) Filed: Mar. 1, 2010

Related U.S. Application Data
(63) Continuation of application No. PCT/EP2008/006858, filed on Aug. 20, 2008.
(60) Provisional application No. 61/015,240, filed on Dec. 20, 2007.

(30) Foreign Application Priority Data
Sep. 5, 2007 (EP) 07291072.2

(51) Int. Cl. A61K 38/44; A61P 9/10

(52) U.S. Cl. 424/94.4

(57) ABSTRACT

The invention relates to the use of a urate oxidase, preferably recombinant urate oxidase, for example rasburicase, for the treatment or prophylaxis of disorders or indirect sequelae of the heart caused by ischemic or reperfusion events, for example during and after cardiac surgery like CABG (coronary artery bypass graft), PCI (percutaneous coronary intervention), transplantation, post myocardial infarction and for the treatment or prophylaxis of coronary artery disease or heart failure, for example congestive heart failure.

### BACKGROUND OF THE INVENTION

The invention relates to the use of urate oxidase, preferably recombinant urate oxidase, for example rasburicase, for the treatment or prophylaxis of disorders or indirect sequelae of the heart caused by ischemic or reperfusion events, for example during and after cardiac surgery like CABG (coronary artery bypass graft), PCI (percutaneous coronary intervention), transplantation, post myocardial infarction and for the treatment or prophylaxis of coronary artery disease or heart failure, for example congestive heart failure.

Uric acid is the end product of purine metabolism in birds, reptiles, primates and humans and is produced in the liver by oxidation of xanthine and hypoxanthine. In all other mammals, uric acid is further oxidized by the enzyme urate oxidase to allantoin. However, humans lack this enzyme. As uric acid has relatively poor water solubility, the increase in plasma levels of uric acid is known to be causative for several diseases such as gout. An acute elevation of uric acid leads to acute renal failure caused by the precipitation of crystals of uric acid in renal tubules (Ejaz A. A. et al., Clin. J. Am. Nephrol. (2007) 2:16-21).

Increase of uric acid production is caused in general in patients suffering from purine metabolism disorders such as hereditable hyperuricaemia. However, acute elevation of high levels of uric acid is also observed in any patient undergoing massive cell death such as during treatment of cancer with cytostatics. The latter is known to lead to the so-called tumor lysis syndrome where massive cell death leads to liberation of nucleic acids being rapidly catabolized into uric acid as the end product due to purine metabolism. In general massive death of cells is also observed in any pathophysiological situation of ischemia and reperfusion and therefore also during cardiac surgery like CABG (coronary artery bypass graft), PCI (percutaneous coronary intervention), transplantation, post myocardial infarction, coronary artery disease or heart failure.

Beside the latter mentioned acute insults, increased plasma uric acid concentrations were recently found to be predictive also for mortality in congestive heart failure (Anker S D et al., Circulation (2003); 107:1991-1997). A causative correlation for this has been also discussed recently (Hare J M et al., Circulation (2003) 107:1951-1953).

Currently three different principles can be used in order to reduce pathophysiological elevated levels of uric acid. (i) Enhancement of renal excretion of uric acid, (ii) impairment of uric acid generation or (iii) conversion of uric acid into allantoin.

### i) Benzbromaron

Treatment with benzbromaron ((2-Ethyl-3-benzofuranyl)-(3,5-dibrom-4-hydroxyphenyl) keton) enhances renal excretion of uric acid by targeting renal uric acid reabsorption. The net effect under benzbromaron treatment is increased excretion of uric acid. Treatment has to begin by subtreshold dosing since benzbromaron itself can trigger the precipitation of uric acid in the kidney or urether.

### ii) Allopurinol

Another approach targets the catabolism of purines into uric acid due to inhibition of xanthinoxidase, a key enzyme in purine metabolism: Allopurinol (4-hydroxypurinol), an analogue of xanthine, is an inhibitor of xanthinoxidase leading to decreased generation of uric acid. Treatment with allopurinol is currently considered the standard pharmacological treatment for hyperuricemia-associated diseases such as gout. During treatment with allopurinol, instead of uric acid, the precursors xanthines accumulate and are mainly excreted via the kidney. Treatment with allopurinol is preventive to avoid high uric acid levels but it is unsuitable in cases of already elevated uric acid levels and is moreover known to induce gout on its own. In case of prevention the tumor lysis syndrome during treatment of cancer, allopurinol is given before cytotoxic treatment. Beside the application of allopurinol, management is directed to normalize metabolic abnormalities and preventing further renal damage.

iii) Urate Oxidase

The mechanism of action of urate oxidase is different from allopurinol. Urate oxidase (uric acid oxidase, urate oxygen oxidoreductase, EC 1.7.3.3) catalyses the oxidation of uric acid to allantoin, a water-soluble product that is easily excreted by the kidney (scheme 1). The protein enzyme urate oxidase can, for example, be obtained from Aspergillus flavus.

The cDNA coding for this protein has been cloned and expressed in Escherichia coli (Legoux R. et al., J. Biol. Chem., 1992, 267, (12), 8565-8570), in Aspergillus flavus (Chevalet L. et al., Curr. Genet., 1992, 21, 447-453) and in Saccharomyces cerevisiae (Leplatois P. et al., Gene., 1992, 122, 139-145).

Recombinant urate oxidase is urate oxidase produced by genetically modified microorganisms and can, for example, be obtained from the above mentioned genetically modified strains of Escherichia coli and Saccharomyces cerevisiae.

Rasburicase is a recombinant urate oxidase enzyme produced from genetically modified strain of Saccharomyces cerevisiae cloned with cDNA from a strain of Aspergillus flavus (Oldfield V et al., Drugs (2006) 66 (4):529-545, Leplatois P. et al., Gene., 1992, 122, 139-145). Rasburicase is a tetrameric protein with identical subunits of a molecular mass of about 34 kDa each (FIG. 1) (SEQ ID NO: 1)—similar to the native Aspergillus flavus urate oxidase (Bayol A. et al., Biotechnol. Appl. Biochem. 2002, 36, 21-31).

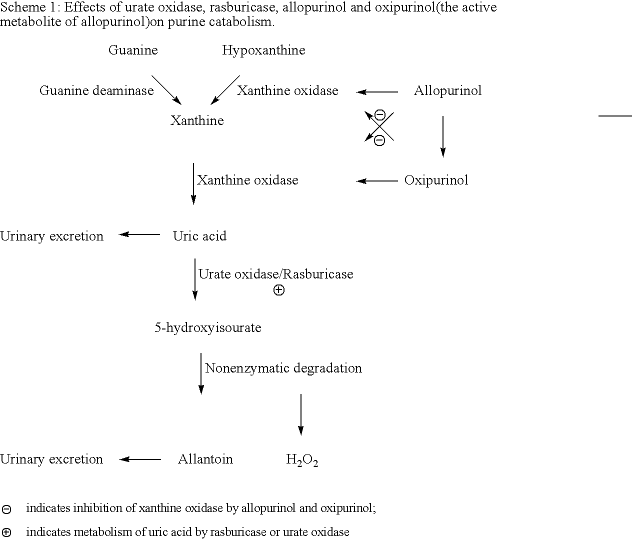

Due to its mode of action, instead of treatment with allopurinol, use of Rasburicase is now the preferred treatment in situations of acute and massively increased plasma uric acid levels in the context of prevention of tumor lysis syndrome.

An apparent disadvantage of urate oxidase treatment is the generation of a stoichiometrically equivalent amount of hydrogenperoxide (Scheme 2), which is according to current knowledge seen as a problem especially in regard of the intended use of urate oxidase in cardiovascular indications.

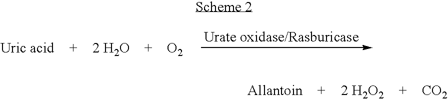

H₂O₂ although not a radical itself, can easily be converted into hydroxyl radicals by Fenton reaction. Different species of endogenously generated oxygen radicals are termed as reactive oxygen species (ROS) comprising also other types such as hydroxylradicals or superoxide anions which are easily converted. Those ROS can be generated by different cellular enzyme systems, for example by NADPH oxidase. In the past ROS were shown to be involved in many physiological and pathophysiological processes. Numerous studies revealed a detrimental role of ROS in regard to cardiovascular indications (Lo S K et al., Am. J. Physiol (1993) 264:L406-412; PMNs; Gasic A C et al., Circulation (1991) November; 84(5): 2154-2166; Bradley J R et al., Am. J. Pathol. (1995); 147(3): 627-641; Kevil C G et al., Am. J. Physiol. Cell Physiol. (2000) July; 279(1): C21-30; Zafari A M et al., Hypertension (1998) September; 32(3): 488-495; for an overview see Cai H, Cardiovascular Research (2005) 68:26-36).

### BRIEF DESCRIPTION OF THE DRAWING

FIG. 1 is a sequence listing of rasburicase, as discussed above.

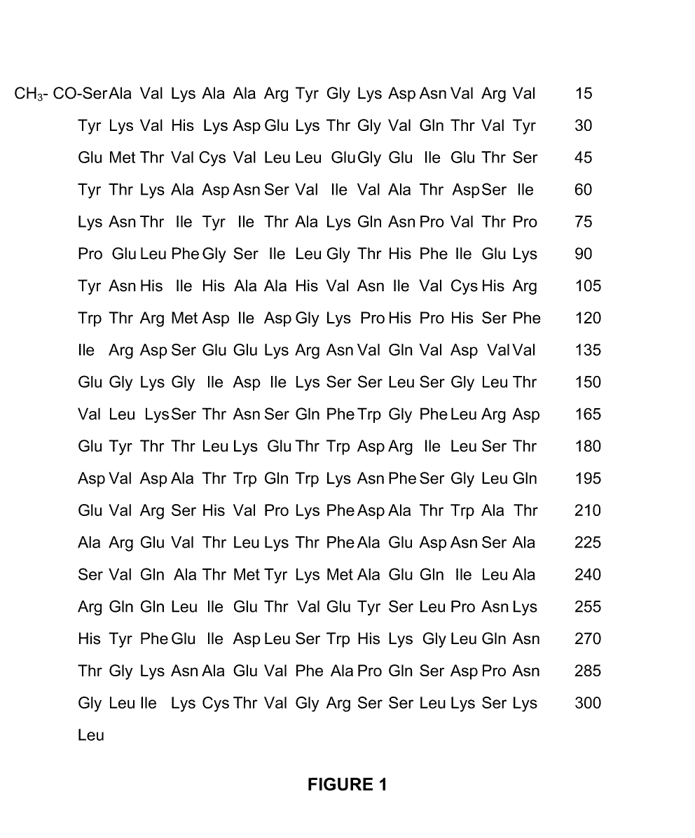

### DESCRIPTION OF THE INVENTION

Experiments have been carried out to test the urate oxidase rasburicase in combination with uric acid for its expected adverse cardiac effects caused by the generated hydrogen peroxide. Suprisingly, the experiments have shown that the heart function is not significantly affected by high concentrations of rasburicase alone or in combination with high concentrations of uric acid. Furthermore, the combination of rasburicase and uric acid even improved heart function and the cardiodynamics when the combination is present prior and during ischemia and reperfusion.

Therefore, the invention relates to the use of a urate oxidase, preferably recombinant urate oxidase, for example rasburicase, for the treatment or prophylaxis of disorders or indirect sequelae of the heart caused by ischemic or reperfusion events, for example during and after cardiac surgery like CABG (coronary artery bypass graft), PCI (percutaneous coronary intervention), transplantation, post myocardial infarction and for the treatment or prophylaxis of coronary artery disease or heart failure, for example congestive heart failure. In another embodiment additional treatment with a scavenger for H₂O₂ is preferred, for example vitamins A, C or E, Trolox, Oligomere Proanthocyanidine, Gluthation, L-N-Acetylcystein, Ebselen, Lycopin, Flavonoid, Catechin and Anthocyan, more preferably L-ascorbic acid.

Pharmaceutical formulations comprise, as an active constituent, an effective dose of rasburicase in addition to customary, pharmaceutically unobjectionable carriers and assistants and optionally also one or more other active pharmacological ingredients, for example ascorbic acid. The pharmaceutical formulations contain normally from 0.1 to 90% by weight of rasburicase.

The pharmaceutical formulations can be produced in a manner known per se. To this end, the active ingredients and/or their physiologically compatible salts, together with one or more solid or liquid pharmaceutical carriers and/or assistants, are converted to a suitable administration form or dosage form, which can then be used as a medicament in human medicine.

Medicaments which comprise rasburicase can be administered, for example, parenterally, intravenously, rectally, nasally, by inhalation or topically, the preferred administration depending on the particular case.

The excipients which are suitable for the desired pharmaceutical formulation are familiar to those skilled in the art on the basis of their expert knowledge. In addition to solvents, gel formers, suppository bases, tablet excipients and other active ingredient carriers, it is possible to use, for example, antioxidants, dispersants, emulsifiers, antifoams, flavorings, preservatives, solubilizers, agents for achieving a depot effect, buffer substances or colorings.

For subcutaneous, intramuscular or intravenous administration, the active compounds used, if desired with the substances customary for this purpose, such as solubilizers, emulsifiers or further excipients, are converted to solution, suspension or emulsion.

Examples of useful solvents are: water, physiological saline or alcohols, for example ethanol, propanol, glycerol, and additionally also sugar solutions such as glucose or mannitol solutions, or else a mixture of the different solvents mentioned.

Examples of suitable pharmaceutical formulations for administration in the form of aerosols or sprays are solutions, suspensions, emulsions or vesicular and micellar medicament forms of the active ingredients or their physiologically compatible salts in water or in a pharmaceutically unobjectionable water-miscible or oily solvent, or a mixture of such solvents. Also suitable for administration in the form of aerosols or sprays, for example for nasal administration, are powders of the active ingredients or their physiologically compatible salts. If required, all formulations may also comprise other pharmaceutical excipients such as isotonizing additives, surfactants, emulsifiers and stabilizers, and also a propellant gas. The formulations mentioned may additionally be in the form of freeze-dried products.

The dosage of rasburicase to be administered in accordance with the invention depends upon the individual case and, for optimal action, should be adjusted to the circumstances of the individual case as usual. For instance, it depends of course upon the frequency of administration and upon the potency and duration of action of the compounds used in each case for treatment or prophylaxis, but also upon the nature and severity of the disease to be treated, and also on the gender, age, weight and individual responsiveness of the human or animal to be treated, and upon whether acute or chronic treatment or prophylaxis is being practiced.

The dosage of rasburicase may typically vary within the range from 1 mg to 1 g per day and per person (at body weight about 75 kg), preferably from 5 to 750 mg per day and person, for example from 100 to 150 mg per day and person. However, higher doses may also be appropriate. The daily dose of the active ingredients may be administered all at once or it may be divided between a plurality of, for example 2, 3 or 4, administrations.

### EXPERIMENTAL PART

### List of Abbreviations

Asc.A Ascorbic acid
kDa Kilo Dalton
n Number of animals
P Pressure
Rasb Rasburicase
Reperf. Reperfusion
UA Uric acid

### Examples of Pharmaceutical Preparations

### Example A

### Aqueous Solution for Intravenous Administration

To prepare 10 ml of solution comprising 50 μg of active compound per ml, 0.5 mg rasburicase were dissolved in 10 ml of isotonic (0.9%) sodium chloride solution.

### Experiments on Isolated Working Rat Hearts

As biological materials the isolated hearts of male Wistar rats were used which were purchased from our Laboratory Animal Science and Welfare (LASW). The heart function (coronary flow and contractility) was investigated on the “Isolated Working Heart” model as previously described (Itter G et al., Laboratory Animals (2005) 39; 178-193). The hearts were first perfused according to Langendorff's method with an oxygenated (95% O₂, 5% CO₂) noncirculating Krebs-Henseleit solution of the following compositions (mmol/L): NaCl, 118; KCl, 4.7; CaCl₂, 2.5; MgSO₄, 1.6; NaHCO₃, 24.9; KH₂PO₄, 1.2; glucose, 5.5; Na-pyruvate, 2.0. A catheter placed into the pulmonary artery drained the coronary effluent perfusate that was collected for determination of coronary flow and venous P_{O}_{₂} measurements. The left atrium was cannulated by an incision of the left auricle. After a 15-minute equilibration period at a fixed perfusion pressure of 60 mmHg, the heart was switched into the working mode at a fixed filling pressure of 11 mmHg. Coronary flow (CF) and pressure signals (dP/dtₘₐₓ) were sampled at 500 Hz, averaged every 2 seconds.

### Effects on Coronary Flow and Contractility of the Heart:

High rasburicase concentrations were tested in combination with different uric acid concentrations on isolated working rat hearts for their possible adverse cardiac effects caused by the generated hydrogenperoxide (H₂O₂).

Table 1 shows that concentrations higher than 100 μM H₂O₂ strongly reduced coronary flow and contractility.

TABLE 1
Effect of increasing H₂O₂ concentrations on coronary flow (CF) and
contractility (dP/dtₘₐₓ) in isolated rat hearts; n = 4, *p < 0.05 vs basal value

| | Basal | H₂O₂ | H₂O₂ | H₂O₂ | H₂O₂ | |
|---|---|---|---|---|---|---|
| | Value | 10 μM | 30 μM | 100 μM | 200 μM | Buffer |
| CF (mL/min) | 14.07 ± 0.33 | 13.89 ± 0.55 | 15.21 ± 1 | 15.7 ± 1.3 | 10.42 ± 0.3* | 14.43 ± 0.96 |
| dP/dtₘₐₓ | 4646 ± 178 | 4774 ± 185 | 4469 ± 167 | 4573 ± 268 | 1698 ± 69* | 2493 ± 182* |
| (mmHg/s) | | | | | | |

Increasing rasburicase concentrations (0.5, 1.5, 5, 15, 50 μg/mL) induced only a slight (not significant) decrease in coronary flow and contractility, which was not influenced in the presence of uric acid (6 mg/L) (Tab. 2). Similar effects were observed when high rasburicase (50 μg/mL) was perfused with higher concentrations of uric acid (6-30 mg/L) (Tab. 3).

TABLE 2
Effect of increasing rasburicase concentrations with and without Uric Acid (UA 6 mg/L)
on coronary flow (CF) and contractility (dP/dtₘₐₓ) in isolated rat heart; n = 6-7/Group

| | Basal | Rasb | Rasb | Rasb | Rasb | Rasb |
|---|---|---|---|---|---|---|
| | Value | 0.5 μg/mL | 1.5 μg/mL | 5 μg/mL | 15 μg/mL | 50 μg/mL |
| CF (mL/min) | 14.39 ± 0.89 | 14.09 ± 0.86 | 13.69 ± 0.89 | 13.59 ± 1.02 | 13.11 ± 0.99 | 12.27 ± 1.16 |
| without UA | | | | | | |
| CF (mL/min) | 15.86 ± 0.79 | 15.7 ± 0.7 | 15.4 ± 0.74 | 15.05 ± 0.68 | 14.62 ± 0.76 | 13.3 ± 0.81 |
| with UA | | | | | | |
| dP/dtₘₐₓ | 4397 ± 259 | 4311 ± 260 | 4271 ± 341 | 4144 ± 267 | 4146 ± 316 | 3917 ± 252 |
| (mmHg/s) | | | | | | |
| without UA | | | | | | |
| dP/dtₘₐₓ | 4534 ± 158 | 4657 ± 215 | 4538 ± 234 | 4491 ± 291 | 4381 ± 292 | 4200 ± 348 |
| (mmHg/s) | | | | | | |
| with UA | | | | | | |

TABLE 3
Effect of increasing Uric Acid and high rasburicase (Rasb 50 μg/mL) concentrations
on coronary flow (CF) and contractility (dP/dtₘₐₓ) in isolated rat hearts; n = 4-5/Group

| | | | Uric Acid | Uric Acid | Uric Acid | |
|---|---|---|---|---|---|---|
| | Basal | Uric Acid | 6 mg/L + | 15 mg/L + | 30 mg/L + | |
| | Value | 6 mg/L | high Rasb | high Rasb | high Rasb | Buffer |
| CF (mL/min) | 12.86 ± 1.56 | 12.86 ± 1.47 | 13.23 ± 1.61 | 12.27 ± 1.54 | 13.34 ± 1.56 | 13.47 ± 1.32 |
| Control | | | | | | |
| CF (mL/min) | 13.32 ± 0.99 | 13.51 ± 1.02 | 12.57 ± 1 | 12.35 ± 1.31 | 11.06 ± 1.22 | 10.58 ± 1.2 |
| with UA | | | | | | |
| dP/dtₘₐₓ | 3550 ± 447 | 3599 ± 524 | 3745 ± 581 | 3845 ± 570 | 3897 ± 455 | 4100 ± 390 |
| (mmHg/s) | | | | | | |
| Control | | | | | | |
| dP/dtₘₐₓ | 3780 ± 213 | 3867 ± 327 | 3821 ± 347 | 3982 ± 341 | 3610 ± 241 | 3671 ± 252 |
| (mmHg/s) | | | | | | |
| with UA | | | | | | |

Effects on Coronary Flow and Contractility of the Heart with Global Ischemia and Reperfusion:

High rasburicase (50 μg/mL) in combination with high uric acid concentrations (15 or 30 mg/L), concentration-dependently improved the recovery after ischemia/reperfusion (Tab. 4, 5).

TABLE 4
Effect of high rasburicase (Rasb 50 μg/mL) and Uric Acid (UA 15 mg/L) concentrations
on coronary flow (CF) and contractility (dP/dtₘₐₓ) in isolated rat hearts with
global ischemia and reperfusion; n = 5/Group; *p < 0.05 vs Control

| | 5 min | 1 min | | Reperf. | Reperf. | Reperf. |
|---|---|---|---|---|---|---|
| | before | before | Global | 5 min after | 10 min after | 15 min after |
| | ischemia | ischemia | Ischemia | Ischemia | Ischemia | Ischemia |
| CF (mL/min) | 13.28 ± 0.7 | 12.82 ± 0.75 | 0.38 ± 0.06 | 4.47 ± 1.69 | 4.71 ± 1.86 | 4.73 ± 1.85 |
| Control with UA | | | | | | |
| CF (mL/min) | 14.55 ± 0.7 | 14.59 ± 0.75 | 0.38 ± 0.01 | 9.76 ± 2.4* | 8.24 ± 2.95 | 7.94 ± 2.83 |
| with UA + Rasb | | | | | | |
| dP/dtₘₐₓ | 3600 ± 250 | 3608 ± 318 | 61 ± 1 | 1057 ± 496 | 1284 ± 667 | 1403 ± 736 |
| (mmHg/s) | | | | | | |
| Control with UA | | | | | | |
| dP/dtₘₐₓ | 4000 ± 270 | 4074 ± 277 | 61 ± 1 | 2987 ± 687* | 2056 ± 672 | 2110 ± 774 |
| (mmHg/s) | | | | | | |
| with UA + Rasb | | | | | | |

TABLE 5
Effect of high rasburicase (Rasb 50 μg/mL) and Uric Acid (UA 30 mg/L) concentrations
on coronary flow (CF) and contractility (dP/dtₘₐₓ) in isolated rat hearts with
global ischemia and reperfusion; n = 5/Group; *p < 0.05 vs Control

| | 5 min | 1 min | | Reperf. | Reperf. | Reperf. |
|---|---|---|---|---|---|---|
| | before | before | Global | 5 min after | 10 min after | 15 min after |
| | ischemia | ischemia | Ischemia | Ischemia | Ischemia | Ischemia |
| CF (mL/min) | 13.33 ± 0.7 | 13.11 ± 0.88 | 0.31 ± 0.02 | 8.34 ± 1.88 | 9.16 ± 1.29 | 9.25 ± 0.63 |
| Control | | | | | | |
| with UA | | | | | | |
| CF (mL/min) | 15.14 ± 0.8 | 15.21 ± 1.06 | 0.39 ± 0.02 | 12.89 ± 2.2* | 12.9 ± 1.35* | 12.44 ± 1.1* |
| with UA + Rasb | | | | | | |
| dP/dtₘₐₓ | 4020 ± 350 | 4017 ± 437 | 59 ± 2 | 2224 ± 418 | 2898 ± 314 | 3192 ± 273 |
| (mmHg/s) | | | | | | |
| Control with UA | | | | | | |
| dP/dtₘₐₓ | 4090 ± 339 | 4115 ± 328 | 61 ± 3 | 3759 ± 587* | 3300 ± 75 | 3373 ± 52 |
| (mmHg/s) | | | | | | |
| with UA + Rasb | | | | | | |

Addition of ascorbic acid (1 mM) led to a normalization of coronary flow and a further improvement on contractility after ischemia/reperfusion (Tab. 6).

TABLE 6
Effect of high rasburicase (Rasb 50 μg/mL), Uric Acid (UA 30 mg/L) and Ascorbic Acid
(Asc.A. 1 mM) concentrations on coronary flow (CF) and contractility (dP/dtₘₐₓ) in isolated
rat hearts with global ischemia and reperfusion; n = 6/Group; *p < 0.05 vs Control

| | 5 min | 1 min | | Reperf. | Reperf | Reperf. |
|---|---|---|---|---|---|---|
| | before | before | Global | 5 min after | 10 min after | 15 min after |
| | ischemia | ischemia | Ischemia | Ischemia | Ischemia | Ischemia |
| CF (mL/min) | 13.84 ± 0.88 | 13.32 ± 0.52 | 0.21 ± 0.02 | 12.85 ± 2.78 | 14.87 ± 0.59 | 13.48 ± 0.57 |
| Control | | | | | | |
| with UA + Rasb | | | | | | |
| CF (mL/min) | 14.34 ± 0.98 | 14.23 ± 1.1 | 0.35 ± 0.02 | 15 ± 1.66 | 13.91 ± 1.7 | 12.64 ± 1.6 |
| with UA + | | | | | | |
| Rasb + Asc.A. | | | | | | |
| dP/dtₘₐₓ | 4813 ± 300 | 4849 ± 314 | 61 ± 2 | 2624 ± 625 | 3730 ± 156 | 4160 ± 255 |
| (mmHg/s) | | | | | | |
| Control | | | | | | |
| with UA + Rasb | | | | | | |
| dP/dtₘₐₓ | 4574 ± 312 | 4437 ± 297 | 58 ± 2 | 3862 ± 379* | 3976 ± 268 | 4002 ± 274 |
| (mmHg/s) | | | | | | |
| Control with UA | | | | | | |
| Rasb + Asc. A. | | | | | | |

As shown above heart function was not significantly affected by high concentrations of rasburicase alone or in combination with high concentrations of uric acid.

Suprisingly, the use of rasburicase in the presence of uric acid even improved heart function when present prior and during ischemia/reperfusion.

In the scenario of cardiac surgery and heart failure, treatment with rasburicase is assumed to be suitable and safe. In our ischemia/reperfusion experiments rasburicase even improved cardiodynamics after ischemia.

What is claimed is:

1. A method for the treatment or prophylaxis of disorders or indirect sequelae of the heart caused by ischemic or reperfusion events, the method comprising administration of a therapeutically effective dose of urate oxidase.

2. The method according to claim 1 for the treatment or prophylaxis of heart failure.

3. The method according to claim 1 for the treatment or prophylaxis of congestive heart failure.

4. The method according to claim 1 for the treatment or prophylaxis of disorders or indirect sequelae of the heart caused by ischemic or reperfusion events during and after cardiac surgery.

5. The method according to claim 1 for the treatment or prophylaxis of disorders or indirect sequelae of the heart caused by ischemic or reperfusion events during and after coronary artery bypass graft, percutaneous coronary intervention, or transplantation.

6. The method according to claim 1 for the treatment or prophylaxis of myocardial infarction.

7. The method according to claim 1, wherein the urate oxidase is a recombinant urate oxidase.

8. The method according to claim 7, wherein the urate oxidase is rasburicase.

9. The method according to claim 1 further comprising treatment with an H₂O₂ scavenger.

10. The method according to claim 9 wherein the H₂O₂ scavenger is ascorbic acid.


SEQUENCE LISTING (1 sequence)

SEQ ID NO: 1 - protein, 301 aa, Aspergillus flavus
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL